(19) United States
(12) Patent Application Publication
(10) Pub. No.: US 2021/0015585 A1
(43) Pub. Date: Jan. 21, 2021

(54) ADHESIVE FOIL MINISCREW GUIDE

(71) Applicant: President and Fellows of Harvard College, Cambridge, MA (US)

(72) Inventors: George E. BORK, Dearborn, MI (US); Mohamed I. Masoud, Newton, MA (US)

(73) Assignee: President and Fellows of Harvard College, Cambridge, MA (US)

(21) Appl. No.: 16/932,045

(22) Filed: Jul. 17, 2020

Related U.S. Application Data
(60) Provisional application No. 62/876,351, filed on Jul. 19, 2019.

(51) Int. Cl. A61C 1/08

(52) U.S. Cl. CPC A61C 1/084; A61C 8/0096; A61C 1/088

(57) ABSTRACT

Herein are described surgical screw guides comprising a radio-translucent portion and a radio-opaque portion. The guides are configured for attachment to tissue of a subject, such as gingiva and provide a method for imaging where to insert miniscrews.

### CROSS-REFERENCE TO RELATED APPLICATIONS

This application claims any and all benefits as provided by law including benefit under 35 U.S.C. § 119(e) of the U.S. Provisional Application No. 62/876,351, filed Jul. 19, 2019, the content of which is incorporated herein by reference in its entirety.

### FIELD OF THE INVENTION

This invention relates to devices and method for providing surgical anchorage. For example, orthodontic anchorage using a miniscrew surgical guide.

### BACKGROUND

Orthodontic anchorage, defined as resistance to unwanted tooth movement, can be provided by teeth, the palate, extraoral structures including the head and neck, or Temporary Anchorage Devices (TADs). Directing reactionary forces to teeth can result in undesirable movement of anchorage units. Redirecting forces to the palate through devices such as a Nance appliance can provide augmented, but not absolute anchorage and can result in intolerable tissue irritation (Kupietzky A, Tal E., The transpalatal arch: an alternative to the Nance appliance for space maintenance. Pediatr Dent. 2007;29(3):235-238). Application of extraoral forces from appliances such as headgear can bolster anchorage and reduce side effects but relies heavily on patient compliance, which is often poor, and provides larger than ideal forces against the teeth (Bos A., Kleverlaan C. J., Hoogstraten J., Prahl-Andersen B., Kuitert R., Comparing subjective and objective measures of headgear compliance. Am J Orthod Dentofac Orthop. 2007;132:801-805). The use of TADs including miniscrew implants (MSIs) can establish absolute anchorage, virtually eliminating unwanted tooth movement and greatly simplifying treatment mechanics (Papadopoulos M. A., Papageorgiou S. N., Zogakis I. P., Clinical effectiveness of orthodontic miniscrew implants: a meta-analysis. J Dent Res. 2011;90(8):969-976). Reduction of these side effects can make orthodontic treatment easier, more efficient, and more predictable. Utilization of absolute skeletal anchorage has expanded what were the previous limitations of orthodontic tooth movement, allowing greater varieties and magnitudes of movement than were previously thought possible. Prior to the introduction of TADs, intrusion of posterior teeth was virtually impossible. Through intrusion of posterior teeth, the occlusal plane can be modified, in terms of both roll and pitch (Takano-Yamamoto T., Kuroda S., Titanium screw anchorage for correction of canted occlusal plane in patients with facial asymmetry. Am J Orthod Dentofac Orthop. 2007;132:237-242). Furthermore, in a patient with a retrusive chin, the chin point can be brought forward through increased jaw closure and associated counter-clockwise autorotation of the mandible (Yao C. C., Lai E. H., Chang J. Z., Comparison of treatment outcomes between skeletal anchorage and extraoral anchorage in adults with maxillary dentoalveolar protrusion. Am J Orthod Dentofac Orthop. 2008;134:615-624). While the use of TADs including MSIs has significantly enhanced the possibilities of orthodontic treatment, many clinicians remain wary of the risks involved and avoid their placement (Hyde J. D., King G. J., Greenlee G. M., Spiekerman C., Huang, G. J., Survey of orthodontists' attitudes and experiences regarding miniscrew implants. J Clin Orthod. 2010;44:481486).

Miniscrew failure and injury to adjacent roots are two potential complications involved in the placement of interradicular MSIs. While minor root injuries can be of limited clinical significance, cracking or fracturing an adjacent root is irreversible and necessitates extraction (Alves Jr M., Baratieri C., Araújo M. T., Souza M. M., Maia L. C., Root damage associated with intermaxillary screws: a systematic review. Int J Oral Maxillofac Surg. 2012;41:14451450; Lee Y. K., Kim J. W., Baek S. H., Kim T. W., Chang Y I. Root and bone response to the proximity of a mini-implant under orthodontic loading. Angle Orthod. 2010;80:452-458). Even minor root contact often results in thermal sensitivity (Maino B G, Mura P., Bednar J., Miniscrew implants: the spider screw anchorage system. Semin Orthod. 2005;11:40-46). When root contact is evident, miniscrews should be removed (Park Y, Lee S. Y., Kim D. H., Jee S. H., Intrusion of posterior teeth using miniscrew implants. Am J Orthod Dentofac Orthop. 2003;123:690-694). Furthermore, root proximity and contact have been associated with an increased risk of mobility and implant failure. Root proximity has been classified as the most critical predictor of miniscrew success, particularly in the mandible (Watanabe H, Deguchi T, Hasegawa M, Ito M, Kim S, Takano-Yamamoto T. Orthodontic miniscrew failure rate and root proximity, insertion angle, bone contact length, and bone density. Orthod Craniofac Res. 2013;16:44-55). Maximum bony stresses under occlusal forces are inversely related to root proximity. With root contact, stresses increase by seven-fold and local bone resorption occurs (Motoyoshi M, Ueno S, Okazaki K, Simizu N. Bone stress for a mini-implant close to the roots of adjacent teeth—3D finite element analysis. Int J Oral Maxillofac Surg. 2009;38:363-368). In a sense, the term “absolute anchorage” is a misnomer. While MSIs do not have periodontal ligament structures (PDLs), studies have indicated that they do not remain completely motionless under orthodontic loads (Liou E. J., Pai B. C., Lin J. C., Do miniscrews remain stationary under orthodontic forces? Am J Orthod Dentofac Orthop. 2004;126:42-47). Because of this, it is advisable to place implants at least 2 mm away from roots on either side of the insertion site to allow for potential miniscrew drifting (Ohashi E., Pecho O., Moron M., Lagravere M., Implant vs screw loading protocols in orthodontics. Angle Orthod. 2006;76:721-727). In addition to root proximity, insertion of MSIs into nonkeratinized tissue can limit success rates due to increased risk of infection and associated inflammatory bone resorption (Herman R., Cope J. B., Miniscrew implants: imtec mini ortho implants. Semin Orthod. 2005;11:32-39). Because of this, MSIs should be placed into attached gingiva whenever possible. Miniscrews should also be inserted away from frenums and muscle attachments to avoid soft tissue impingement and to allow for good oral hygiene, reducing peri-implant inflammation and bone loss (Miyawaki S., Koyama I., Inoue M., Mishima K., Sugahara T., Takano-Yamamoto T., Factors associated with the stability of titanium screws placed in the posterior region for orthodontic anchorage., Am J Orthod Dentofac Orthop. 2003;124:373-378).

Insertion angle is another important consideration in miniscrew placement. Insertion of MSIs perpendicular to the bony surface is not always indicated due to the high risk of root injury. Roots tend to diverge as they move apically. Because of this, it is recommended that miniscrews be inserted obliquely, in an apical direction. Angles of 30°-40° have been recommended in the maxilla while angles of 10°-20° have been recommended in the mandible. The exception is for MSIs approximating the maxillary sinus, where it is advisable to place implants perpendicular to the bony surface to limit risk of perforating the sinus floor (Papadopoulos M. A., Tarawneh F., The use of miniscrew implants for temporary skeletal anchorage in orthodontics: a comprehensive review. Oral Surg Oral Med Oral Pathol Oral Radiol Endod. 2007;103:e6-e15). Oblique insertion of miniscrews also effectively limits maximum stresses under occlusal loads and allows for insertion of longer miniscrews in areas of limited alveolar ridge width (Suzuki A, Masuda T, Takahashi I, Deguchi T, Suzuki O, Takano-Yamamoto T. Changes in stress distribution of orthodontic miniscrews and surrounding bone evaluated by 3-dimensional finite element analysis. Am J Orthod Dentofac Orthop. 2011;140:e273-280). While early failure of MSIs leaves clinicians with the option to modify the treatment plan or mechanical approach, a majority of MSI failures come 100-150 days after placement, making changes to treatment plans at that point difficult to impossible (Wiechmann D, Meyer U, Büchter A. Success rate of mini- and micro-implants used for orthodontic anchorage: a prospective clinical study. Clin Oral Implants Res. 2007;18:263267).

To ease clinician anxiety, limit root proximity, and reduce the incidence of root injury, several designs of miniscrew placement guides have been developed. These guides range from as simple as a bent orthodontic wire to as complex as 3D printed stents based on cone beam computed tomography.

Many of these guides have been demonstrated to be effective in reducing interoperator variability, increasing placement accuracy, limiting root proximity, and preventing iatrogenic root injury. However, existing miniscrew surgical guides are insufficient due to their bulky intraoral size, difficulty in fabrication, necessity for additional CBCT images, and limits in accurately fabricating guides based on these 3D images (Bae M J, Kim J Y, Park J T, Cha J Y, Kim H J, Yu H S, Hwang C J. Accuracy of miniscrew surgical guides assessed from cone-beam computed tomography and digital models. Am J Orthod Dentofac Orthop. 2013;143:893-901). Additionally, printed stents require additional appointments to be scheduled to allow for printing time, costing patient time and doctor time in addition to the cost of materials.

There therefore remains a need for miniscrew surgical guides that are easy to use, easy to mass produce and do not require significant addition imaging and specialized fabrication. These anchorage methods should be effective in reducing interoperator variability, increasing placement accuracy, limiting root proximity, and preventing iatrogenic root injury.

### SUMMARY

In general, this disclosure relates to surgical anchorage methods and devices. More particularly, this disclosure, in some aspects is directed to orthodontic anchorage.

In a first aspect this disclosure is of a surgical guide comprising a radio-translucent member extending in a first direction along a first axis from a first end to a second end. The radio-translucent member including a channel extending along the first axis from the first end to the second end, and configured for guiding a screw therethrough. The radio-translucent member also including a radio-opaque portion adhered to the first end, wherein the radio-opaque portion includes an opening that is aligned with the channel. Optionally, the surgical guide further comprises a tab consisting of a radio-translucent material attached between the first end of the elongated member and the radio-opaque portion.

Optionally, the surgical screw guide comprises at least three flat surfaces defining the channel and configured to engage screw flights of said screw for said guiding of the screw therethrough. Optionally, the channel is a closed channel defined by the at least three flat surfaces, the closed channel having an opening at the first end and an opening at the second end.

Optionally, the surgical screw guide further comprising an adhesive covering at least a portion of a flat exterior surface of the radio-opaque portion, wherein the adhesive and flat surface are configured for attachment of the radio-opaque portion to biological tissue. Optionally wherein the adhesive is an orthodontic grade adhesive.

In some options the surgical screw guide is further comprising a slit extending through the length of the surgical screw guide in the first direction and wherein the slit extends through an outer surface of the radio-translucent member to the channel.

Optionally, the tab is configured as a flat member having a first surface attached to the first end of the radio-translucent member, a second surface attached to the radio-opaque portion, an opening that is aligned with the channel, and wherein the tab is configured not to adhere to biological tissue. Optionally, the radio-opaque portion includes a flat outer surface that is oriented at an angle relative to the first direction selected from the group consisting of about 90 degrees, about 105 degrees and about 125 degrees.

Optionally, the radio-opaque portion attenuates more radiation than the rest of the radio-translucent member not including the radio-opaque portion, wherein the radiation is provided from a radiation beam oriented to irradiate in a direction approximately perpendicular to a flat outer surface of the radio-opaque portion and pass through the radio-translucent member. Optionally, the radio-opaque portion has a Hounsfield Unit (HU) greater than the HU of the radio-translucent member that is not the radio-opaque portion. For example, optionally the radio-opaque portion has a HU number above about 100. Optionally, the radio-translucent member comprises a silicone polymer or an organic polymer. Optionally, the radio-opaque portion comprises a metal. For example, optionally wherein the metal is a stainless steel. Optionally, the radio-opaque portion is a stainless steel foil having a thickness between about 0.001 and 0.01 inches configured wherein the opening has a perimeter in the shape of a cylinder cross section.

Optionally, the surgical screw guide further comprises a miniscrew threaded into the channel, wherein the miniscrew has a diameter between 1 and 2 mm, and a length between 4 and 12 mm.

In a second aspect this disclosure is of a surgical guide comprising a tube extending along a primary axis and having a first end and a second end and a channel extending between the first end and second end along the primary axis. The tube comprising at the first end of a thin foil and a tab extending in a direction between about 0 and about 35 degrees of a perpendicular to the primary axis, wherein the thin foil comprises a radio-opaque material and the tube not including the thin foil comprises a radio-translucent material. The tube also comprising a slit between the first end and second end and extending through the tube to the channel and through the tab. Optionally, the thin foil includes an external surface for adhesion to biological tissue, and the external surface is substantially flat, the external surface oriented at an angle that is between about 90 degrees and about 125 degrees with respect to the primary axis. Also optionally, the channel is configured for guiding a surgical miniscrew therethrough.

In a third aspect, this disclosure relates to a surgical guide comprising a radio-opaque foil comprising a first surface and a second surface and an opening in the foil connecting the first surface and second surface, wherein at least a portion of the first surface comprises a film of orthodontic grade adhesive, and wherein the opening is configured for threading a miniscrew therethrough. Optionally the surgical guide further comprising a radio-translucent tube extending along a primary axis and having a first end and a second end and a channel extending between the first end and second end along the primary axis, wherein the first end of the tube comprises a third surface and said third surface is attached to the second surface of the radio-opaque foil, wherein the opening in the foil is aligned to the channel. Optionally the surgical guide further comprises a tab constructed of a radio-translucent material attached to the second surface and extending outwards from the foil in a direction parallel to the first surface.

In a fourth aspect, this disclosure relates to a method for providing orthodontic anchorage. The method comprising attaching a surgical guide to a subject, wherein the surgical guide comprises the surgical guide according to the first aspect, second aspect or third aspect of the disclosure. The method further comprising irradiating the radio-opaque portion with an X-ray beam from an X-ray system, wherein the X-ray beams is oriented approximately perpendicular to the flat outer surface of the radio-opaque portion, and detecting and processing x-rays transmitted through the radio-opaque portion using the X-ray system to provide an image of the radio-opaque portion relative to the teeth and jaw bone of said subject. The method further comprises feeding a screw through the screw guide of the surgical guide and fastening the screw into the jaw bone, and then detaching and removing the surgical screw guide from the patient.

Miniscrew surgical guides as described herein provide several advantages. For example, the guides are easy to use so that they can reduce interoperator variability. In addition, materials for their construction can be economically sourced and manipulated.

These and other capabilities of the inventions, along with the inventions, will be more fully understood after a review of the following figures, detailed description, and claims.

### BRIEF DESCRIPTION OF THE FIGURES

The accompanying drawings, which are incorporated into this specification, illustrate one or more exemplary embodiments of the inventions and, together with the detailed description, serve to explain the principles and applications of these inventions. The drawings and detailed description are illustrative, and are intended to facilitate an understanding of the inventions and their application without limiting the scope of the invention. The illustrative embodiments can be modified and adapted without departing from the spirit and scope of the inventions.

FIG. 1 shows an embodiment of a surgical guide and screw for placement of a miniscrew.

FIG. 2A is a top view of the guide and screw shown in FIG. 1.

FIG. 2B is a top view of another embodiment of a surgical guide and screw for placement of a miniscrew.

FIG. 2C is a top view of yet another embodiment of a surgical guide and screw for placement of a miniscrew.

FIG. 3A shows an embodiment of a Maxillary Sinus Guide.

FIG. 3B shows an embodiment of a Mandibular Guide.

FIG. 3C shows and embodiment of a Maxillary Guide.

FIG. 4A is a diagrammatic depiction of an irradiation of a surgical guide according to one embodiment.

FIG. 4B is a diagrammatic depiction of an irradiation of a surgical guide according to another embodiment.

FIG. 5 is X-ray image of a guide on a subject.

### DETAILED DESCRIPTION

The present disclosure relates surgical implants, and methods for placing these implants. For example, surgical implants that provide orthodontic anchorage.

In orthodontics, forces are intentionally applied to teeth to produce desired tooth movements. The corresponding reactionary forces often promote undesired side effects, including counterproductive movement of other teeth. Anchorage, in the orthodontic sense, refers to resistance to these reactionary forces and their associated effects on tooth position. Because physiologic tooth movement is driven by the periodontal ligament (PDL), structures without active PDLs (ankylosed teeth, skeletal implants, etc.) will not move when exposed to orthodontic forces. Directing reactionary forces to these immovable structures can effectively prevent unwanted side effects of orthodontic forces. This concept is referred to as absolute anchorage. Temporary anchorage devices (TAD) allow practitioners to move teeth with absolute anchorage, avoiding deleterious side effects including unwanted tooth movement. Mini screw implants (MSI) are a form of temporary anchorage devices used in orthodontic practice.

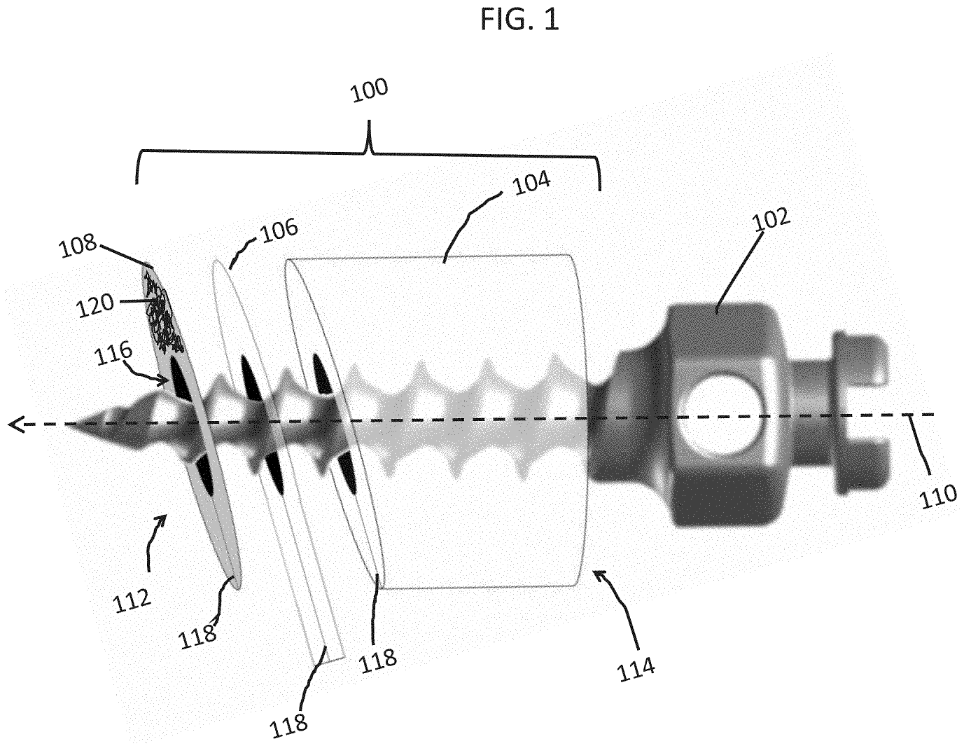

An embodiment of a surgical guide 100, such as can be used for placement of a miniscrew 102, is shown by reference to FIG. 1. In this figure, the guide is shown with components in a blown up view. The guide comprises components 104, 106, and 108 that extend in a first direction along a first axis 110, (indicated with the dotted arrow) from a first end 112, to a second end 114. The guide 100 also includes a channel 116 which is configured for guiding the screw (e.g., a miniscrew) 102 therethrough. Components 104 and 106 are radio-translucent, while component 108 is radio-opaque. Component 106 is configured as a tab (e.g., a pull tab) and extends out from components 104 and 112.

In some embodiments component 108 can be used as a surgical guide without components 106 and 104. That is, the guide is configured as a foil that has an opening therethrough. In some other embodiments the guide includes components 108 and 104 but does not include tab 106.

In some embodiments the surgical guide includes a slit 118 that extends through the length of the surgical guide, as shown in FIG. 1 in components 104, 106, and 108. The slit can comprise of a series of regular cuts or holes (e.g., a perforation) or irregular cuts or holes. The purpose of the cut is to weaken the structure so that the entire structure can be broken apart for removal as will be described in more detail below.

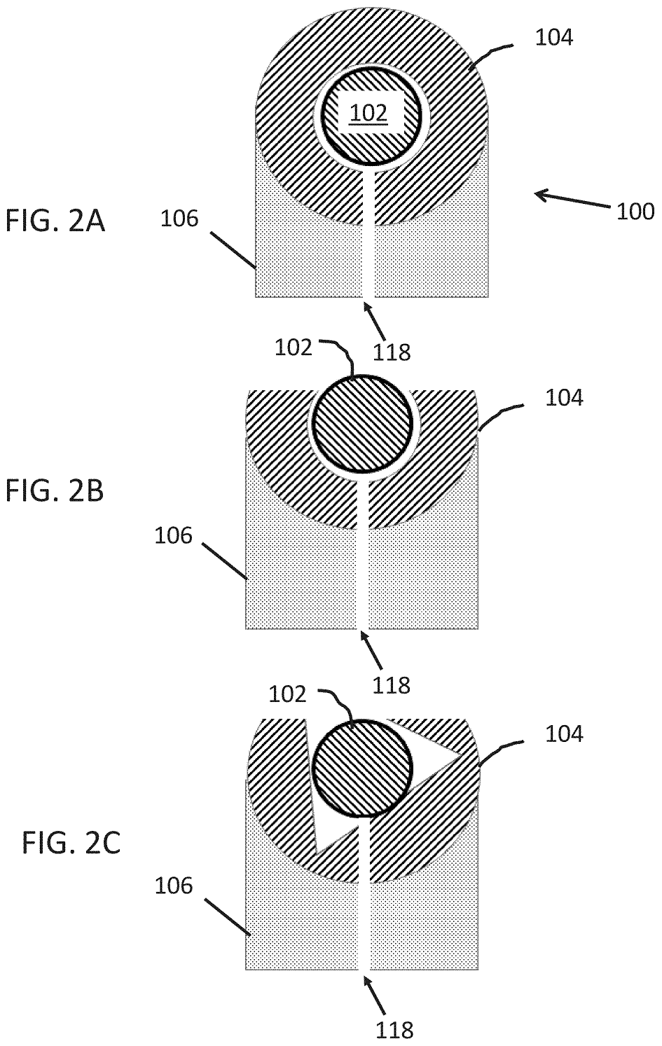

FIG. 2A is a top view of the guide 100 showing the component 104 and a screw 102. For clarity, the screw head of 102 is not shown. The slit 118 is shown as removed material, although as noted above, the slit may be a cut through the material (and may also only be a partial removal of material or cut, such as a perforation). The slit 118 is shown to be aligned throughout the components. In some embodiments the slit can be curved or helical through the structure.

In some embodiments the channel is a cylinder or cylindrical in the shape as shown in FIG. 1 and in FIG. 2A. In other embodiments the channel is not a closed cylinder but configured, for example, as an open trough or conduit with curved surfaces contacting the screw shaft as shown in FIG. 2B. In some other embodiments, as depicted in FIG. 2C the channel is configured with flat surfaces. Although the contact between the screw shaft and channel surface is not always shown in these depiction, it is understood that this contact occurs so that the channel can guide the screw through the channel. In some embodiments, as depicted in FIG. 2C the shaft of the screw is contacted with at least three surfaces for guiding the screw. The channel can also include features to engage the flights of the screw. For example, the channel can be tapped with grooves to match the screw flights.

In some embodiments the outer surface of radio-opaque portion 108 is covered with an adhesive 120. The adhesive is shown, in FIG. 1, only covering a portion of the outer surface of 108 for clarity although it is understood that a portion of the entire surface, or the entire outer surface can be covered by this adhesive on the surface opposite the surface in contact with the tab 106. For example, the adhesive can be spread on the surface to form a film, such as a film covering at least a portion of this surface. In some embodiments the outer surface is configured to match the approximate curvature of tissue to which it will be attached to. For example, the surface is approximately flat, such as having a radius of curvature between about 10 cm convex and 10 cm concave. The radius of curvature can also vary from concave to convex over the entire surface.

In some embodiments the adhesive 120 is an adhesive that when it is applied to 100 and then contacted with a tissue of a subject, the bond formed is strong enough to resist movement or detachment from the tissue. For example, in some embodiments the adhesive is used to attach the guide 100 to gingiva of a subject and the adhesive forms a bond that is strong enough to resist movement of or detachment due to incidental contact with the cheeks of the subject. In some embodiments the adhesive is safe to use in an oral environment. In some embodiments the adhesive is a cyanoacrylate or the like. In some embodiments the adhesive is Orabase® Soothe-N-Seal™ (Colgate), or a similar adhesive that can also be used for aphthous ulcers. In some embodiments the adhesive is radio-translucent. In other embodiments the adhesive is radio-opaque. In some embodiments, the adhesive is radio-opaque and replaces the radio-opaque portion or is the radio-opaque portion of 100. For example, the adhesive is applied directly to the tab 106. For example, the adhesive can be a mixture of an adhesive, such as a cyanoacrylate, and a radio-opaque material (e.g., such as a material of radio-opaque particles).

As used herein “radio-translucent” and “radio-opaque” relates to the degree to which radiation such X-rays are transmitted or penetrate through a material. For example, a radio-opaque material is opaque to radiation, such as X-rays. The opposite of radio-opaque is radio-translucent. As used herein, a radio-translucent component will transmit more radiation therethrough than a radio-translucent material. In some embodiments a radio-opaque component will attenuate X-rays to a larger degree than a radio-translucent component. An X-ray attenuation coefficient is a measure of how easily a material can be penetrated by radiation such as an X-ray beam. It quantifies how much the beam is attenuated (e.g., weakened) by the material it is passing through.

A unit for quantification of material with respect to radio-translucence and radio-opaqueness is the “Hounsfield unit.” Hounsfield units (HU) are a dimensionless unit used, for example, in computed tomography (CT) scanning to express CT numbers in a standardized and convenient form. Hounsfield units are obtained from a linear transformation of the measured attenuation coefficients, where the HU unit for water is set at 0. Hounsfield units span values between about −1000 (air) and about 30,000 (upper limit). For example, HU for tissues can be between −800˜200, while bone can have values between 200 and about 3000.

In some embodiments the radio-opaque components have a HU number greater than about 100, such as greater than about 200, or greater than 300. For example, in some embodiments, the radio-opaque components have HU values between about 300 and the upper limit of measurable radiodensity (such as about 30,000).

In some embodiments, the radio-translucent components have a HU number that is less than the HU number of the radio-opaque member, such as less than about 300, less than about 200, less than about 100, less than about 0, less than about −100, less than about −200, or less than about −300.

In some embodiments radio-translucent components include elements with low Z numbers such a Z numbers below about 20. Some examples include polymers selected from organic polymer and poly siloxanes. Without limitation these can include any polymer selected from the group consisting of low-density polyethylene, high-density polyethylene, polypropylene, polystyrene, polytetrafuloroehtylene, polyvinyl chloride, polychlorotrifluoroethylene, epoxy, cellulose, phenol-formaldehyde resin, para-aramid fiber, para-aramid, polyethylene terephthalate, polyurethane, polychloroprene, polyamide, meta-aramid polymer, polyacrylonitrile (PAN), polytetrafluoroethylene, polyimide, aromatic polyesters, poly-p-phenylene-2,6-benzobisoxazole, silicones, poly dialky silicones, carbon fibers, mixtures of these and copolymers of these. In some embodiments the radio-translucent material is a silicon. In some embodiments the radio-translucent material is a polyester or a polyethylene terephthalate such as Maylar ® (Dupont Tejjin Films). In some embodiment the tab 106 comprises a Mylar® film.

In some embodiment the radio-opaque components include elements with high Z numbers such as above about 20. For example, the radio-opaque materials can include metals selected from the group consisting of iron, nickel, copper, chromium, and zinc, combinations of these including amalgams. In some embodiments the metal is chosen to be a corrosion resistant metal such as a stainless steel.

In some embodiments the radio-opaque component or portion 108 is configured as a flat foil having a thickness of between about 0.001 inches (0.0254 mm) and about 0.01 inches (0. 254 mm) thick. For example, between about 0.05 mm and about 0.2 mm such as between about 0.1 and about 0.15 mm. For example, the component 108 can be a disk made from a stainless steel foil with a hole with a perimeter having a circular cross section. In some embodiments where the material is thick or made of a difficult to tear material, a perforation or slit as described with reference to 118 in FIG. 1 is included. In other embodiments, where the material is thin or made of a less difficult to tear material a perforation is not used.

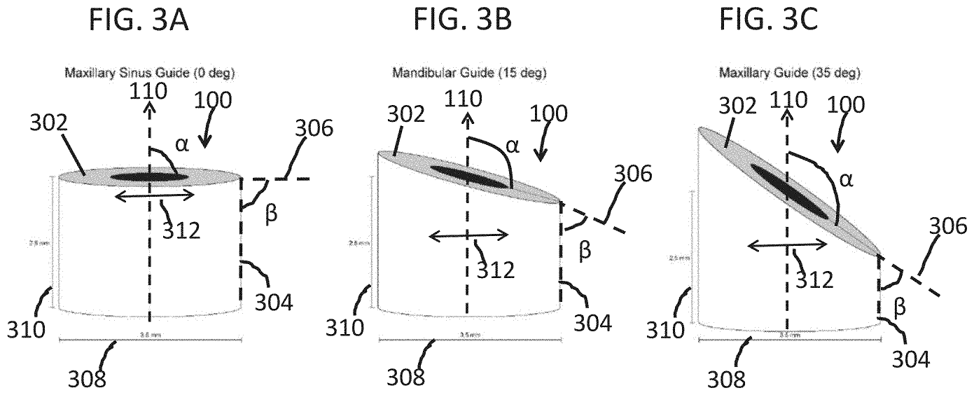

FIG. 3A, 3B and 3C show some embodiments of the guide 100. These are configured as tubes that are cross sectioned to produce the outer surface 302, which is the same surface to which an adhesive 120 (FIG. 1) can be applied, that is approximately flat as previously described, and at specified angles with respect to the direction 110. The tab 106 is not shown in these figures. The maximum angle between a line 306 on the surface 302 and the direction vector 110 is defined as angle α. This angle α can be of any value. In some embodiments the guide 100 is configured to be a Maxillary Sinus Guide (0°, or α−90) as shown by FIG. 3A where α is about 90°, such as between about 85 and 95°, between about 86 and 94, between about 87° and about 93°, between about 88° and 92° or between about 89° and 91°. In some embodiments the guide 100 is configured to be a Mandibular Guide (15°, or α−90) as shown by FIG. 3B and α is about 105°, such as between about 100° and about 110°, such as between about 101° and about 109°, such as between about 102° and about 108°, such as between about 103° and about 107°, such as between about 104° and about 106°. In some embodiments the guide 100 is configured to be a Maxillary Guide (35°, or α−690) as shown by FIG. 3C and α is about 125°, such as between about 120° and about 130°, such as between about 121° and about 129°, such as between about 122° and about 128°, such as between about 123° and about 127°, such as between about 124° and about 126°. Although not shown, for embodiments that include a tab configures as a flat film, such as show in FIG. 1, the film extends from the surface defined by angle β which is complementary to angle α and can be defined by the line 304 parallel to a surface of 100 (as indicated) and a line 306 (as indicated). As used herein “Guide” should not be construed as implying that the guide will direct the mini-screw to, for example the Maxillary Sinus, but rather to provide the recommended angles such as 30°-40° for the maxilla and 10°-20° in the mandible. The angles can be used in order to avoid root damage by the mini-screw placement.

In some embodiments the guide 100 has a diameter 308 of about 3.5 mm (e.g., a diameter between about 2 mm and about 5 mm, between about 2.5 mm and about 4.5 mm, between about 3 mm and about 4 mm). In some embodiments the guide 100 extends from the first end 114 to the second end 112 by a height 310 of about 2.5 mm (e.g., a height between about 2 mm and about 5 mm, between about 2.5 mm and about 4.5 mm, or between about 2.5 mm and about 3.5 mm). Herein, the measurement of height 310 is taken at the first end at the point where a screw that is inserted through the guide 100 would first contact a tissue when the guide is being used. That is, the center of the channel 116 at the first end 114. In some embodiment the inner diameter 312 or a diameter of a circle circumscribed by three surfaces defining the channel (as previously described with reference to FIG. 2C) is about 1.5 mm, for accommodation of a screw having a diameter about 0.1 mm smaller (e.g., about 1.4 mm). For example, the inner diameter can be between about 1.0 mm and about 2.0 mm, such as between about 1.1 and about 1.9 mm, between about 1.2 mm and about 1.8 mm, between about 1.3 mm and about 1.7 mm, between about 1.4 mm and about 1.6 mm. In some embodiments the guide is configured for guiding a screw having a length between about 4 mm and about 12 mm, such as between about 5 and about 10 mm.

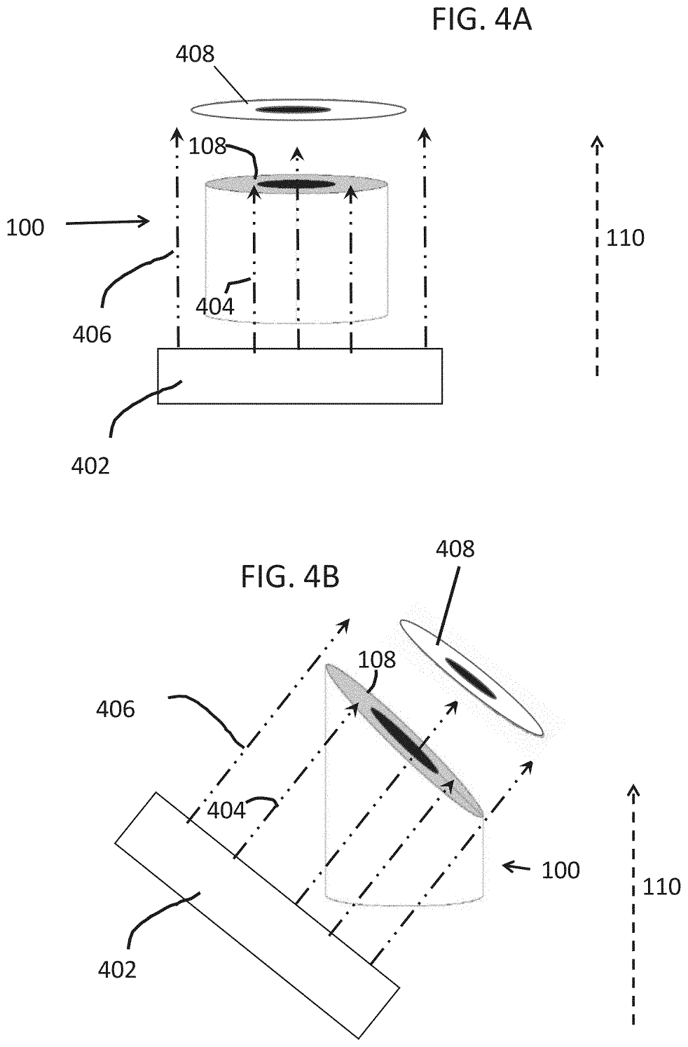

In some embodiments the guide 100 is used to produce an image by irradiation with an X-ray instrument. For example, the guide 100 can be attached to the gingiva of a subject and an X-ray beam from an X-ray instrument is used to irradiate the subject so that the beams are approximately perpendicular to the flat outer portion of the radio-opaque portion. The orientation of the irradiating beam is shown with reference to FIG. 4A and FIG. 4B. A beam comprising of X-rays produces by an X-ray device having an X-ray source 402 and directed to irradiate the guide 100 so that the orientation of the beams is about perpendicular to the radio-opaque component 108. As shown, the orientation is not the same as the orientation of the direction 110. Where X-rays 404 imping on the 108 they are attenuated, for example, being completely or partially (e.g., at least 10%, at least 20%, at least 30%, at least 40%, at least 50%, at least 60%, at least 70%, at least 80%, at least 90% attenuated) blocked from passing therethrough. Where the X-rays 406 do not impinge on 108 they can pass therethrough un-attenuated (e.g., less than about 10% attenuated). Therefore, the beams can outline or produce a reverse image 408 which can be detected.

X-ray images can be produced using X-ray systems. For example, a system can include conventional dental radiograph instruments including X-ray generation device, shielding, detectors, and computers, used for producing dental radiographs. In some embodiments, periapical radiography techniques can be used to image the teeth and guide. In some embodiments an intraoral radiograph is generated to view the area of interest and any intraoral machine or system can be used. In some embodiments NOMAD KaVo NOMAD™ Pro2 portable X-ray device (Aribex) can be used. In some embodiments a non-portable X-ray device can be used.

After the imaging is completed, and the guide is determined to be properly placed, a surgical miniscrew is inserted through the channel 116 of the guide 100 and into the subject (e.g., through the gingiva and into bone) to provide the required anchorage. The guide can then be removed by pulling on the tab 106. The slit 118 can aid in the removal because it provides an easy fracturing of the entire guide. The screw can be further driven into the subject if necessary.

In some optionally embodiments the surgical screw guides can be adapted and used for surgical anchorage other than orthodontic anchorage. For example, and without limitation, the surgical screw guides can be used for the location and placement of surgical screws for fastening and of implants (e.g., titanium, steel or other implants), such as knee or hip implants or for location and placement of surgical screws to temporarily hold together broken bones.

Embodiments of various aspects described herein can be defined as in any of the following numbered paragraphs:
1. A surgical screw guide comprising,
   a radio-translucent member extending in a first direction along a first axis from a first end to a second end,
   the radio-translucent member including a channel extending along the first axis from the first end to the second end, and configured for guiding a screw therethrough,
   the radio-translucent member including a radio-opaque portion adhered to the first end, wherein the radio-opaque portion includes an opening that is aligned with the channel.
2. The surgical guide according to paragraph 1, further comprising a tab consisting of a radio-translucent material attached between the first end of the elongated member and the radio-opaque portion.
3. The surgical guide according to paragraph 2, wherein the tab is configured as a flat member having a first surface attached to the first end of the radio-translucent member, a second surface attached to the radio-opaque portion, an opening that is aligned with the channel, and wherein the tab is configured not to adhere to biological tissue.
4. The surgical screw guide according to any one of paragraphs 1-3, wherein the surgical screw guide comprises at least three flat surfaces defining the channel and configured to engage screw flights of said screw for said guiding of the screw therethrough.
5. The surgical screw guide according to any one of paragraphs 1-4, wherein the channel is a closed channel defined by the at least three flat surfaces, the closed channel having an opening at the first end and an opening at the second end.
6. The surgical screw guide according to any one of paragraphs 1-5, further comprising an adhesive covering at least a portion of a flat exterior surface of the radio-opaque portion, wherein the adhesive and flat surface are configured for attachment of the radio-opaque portion to biological tissue.
7. The surgical screw guide according to paragraph 6, wherein the adhesive is an orthodontic grade adhesive.
8. The surgical screw guide according to any one of paragraphs 1-7, further comprising a slit extending through the length of the surgical screw guide in the first direction and wherein the slit extends through an outer surface of the radio-translucent member to the channel.
9. The surgical screw guide according to any one of paragraphs 1-8, wherein the radio-opaque portion includes a flat outer surface that is oriented at an angle relative to the first direction selected from the group consisting of about 90 degrees, about 105 degrees and about 125 degrees.
10. The surgical screw guide according to any one of paragraphs 1-9, wherein the radio-opaque portion attenuates more radiation than the rest of the radio-translucent member not including the radio-opaque portion, wherein the radiation is provided from a radiation beam oriented to irradiate in a direction approximately perpendicular to a flat outer surface of the radio-opaque portion and pass through the radio-translucent member.
11. The surgical screw guide according to any one of paragraphs 1-10, wherein the radio-opaque portion has a Hounsfield Unit (HU) greater than the HU of the radio-translucent member that is not the radio-opaque portion.
12. The surgical screw guide according to any one of paragraph 11, wherein the radio-opaque portion has a HU number above about 100.
13. The surgical screw guide according to any one of paragraphs 1-12, wherein the radio-translucent member comprises a silicone polymer or an organic polymer.
14. The surgical screw guide as in claim 1, wherein the radio-opaque portion comprises a metal.
15. The surgical screw guide according to any one of paragraph 14, wherein the metal is a stainless steel.
16. The surgical screw guide according to any one of paragraphs 1-15, wherein the radio-opaque portion is a stainless steel foil having a thickness between about 0.001 and 0.01 inches configured wherein the opening has a perimeter in the shape of a cylinder cross section.
17. The surgical screw guide according to any one of paragraphs 1-16, further comprising a miniscrew threaded into the channel, wherein the miniscrew has a diameter between 1 and 2 mm, and a length between 4 and 12 mm.
18. A surgical guide comprising; a radio-opaque foil comprising a first surface and a second surface and an opening in the foil connecting the first surface and second surface, wherein at least a portion of the first surface comprises a film of orthodontic grade adhesive, and wherein the opening is configured for threading a miniscrew therethrough.
19. The surgical guide according to paragraph 18, further comprising a radio-translucent tube extending along a primary axis and having a first end and a second end and a channel extending between the first end and second end along the primary axis, wherein the first end of the tube comprises a third surface and said third surface is attached to the second surface of the radio-opaque foil, wherein the opening in the foil is aligned to the channel.
20. The surgical guide according to paragraph 18 or 19, further comprising a tab constructed of a radio-translucent material attached to the second surface and extending outwards from the foil in a direction parallel to the first surface.
21. A method for providing orthodontic anchorage, the method comprising: attaching a surgical guide to a subject;
   the surgical guide comprising a radio-opaque foil comprising a first surface and a second surface and an opening in the foil connecting the first surface and second surface,
   wherein attaching the surgical screw guide to the subject comprises contacting a gingiva of the subject with an adhesive, and contacting the adhesive with the first surface, and allowing the adhesive to cure;
irradiating the radio-opaque foil with an x-ray beam from an x-ray system, wherein the x-ray beams is oriented approximately perpendicular to the first and second surface of the foil;
detecting and processing x-rays transmitted through the radio-opaque foil using the x-ray system to provide an image of the radio-opaque foil relative to the teeth and jaw bone of said subject; and
feeding a screw through the screw guide of the surgical guide and fastening the screw into the jaw bone.
22. A method for providing orthodontic anchorage, the method comprising:
   attaching a surgical screw guide to a subject,
      the surgical screw guide comprising, a radio-translucent member extending in a first direction along a
      first axis from a first end to a second end, the radio-translucent member including a channel extending along the first axis from the first end to the second end, and configured for guiding a screw therethrough,
      the radio-translucent member including a radio-opaque portion adhered to the first end, wherein the radio-opaque portion includes an opening that is aligned with the channel, and
      a tab consisting of a radio-translucent material attached between the first end of the elongated member and the radio-opaque portion,
      wherein attaching the surgical screw guide to the subject comprises contacting a gingiva of the subject with an adhesive, and contacting the adhesive with a flat outer surface of the radio-opaque portion, and allowing the adhesive to cure;
   irradiating the radio-opaque portion with an x-ray beam from an x-ray system, wherein the x-ray beams is oriented approximately perpendicular to the flat outer surface of the radio-opaque portion;
   detecting and processing x-rays transmitted through the radio-opaque portion using the x-ray system to provide an image of the radio-opaque portion relative to the teeth and jaw bone of said subject;
   feeding a screw through the screw guide of the surgical guide and fastening the screw into the jaw bone;
   detaching and removing the surgical screw guide from the patient.

The embodiments will be more readily understood by reference to the following examples, which are included merely for purposes of illustration of certain aspects and embodiments of the present invention, and should not be construed as limiting. As such, it will be readily apparent that any of the disclosed specific constructs and experimental plan can be substituted within the scope of the present disclosure.

### EXAMPLES

An adhesive foil ring with an obliquely-angled guide tube, applied to attached gingiva at the site of likely insertion was made. Once in place, a periapical radiograph can indicate whether the selected location is sufficiently distant from the roots of adjacent teeth. The use of such a surgical guide can significantly reduce technique sensitivity, improve interoperator variability, decrease root proximity, and eliminate root injuries associated with placement of MSIs.

Significance

The guide changes the standard of care for placement of orthodontic miniscrews. Currently, the standard of care for placement of orthodontic miniscrews is selecting an insertion site based on a combination of clinical and radiographic assessment. Most clinicians utilize a simple periapical radiograph of the surrounding teeth to determine morphology of adjacent roots and to identify available bone. These radiographs usually do not include a position indicator. Because of this, miniscrew implant failure rates remain relatively high and root injuries are not uncommon. The guide as described herein has the potential to make implant placement much safer.

The guide can be mass produced. Existing guides, while shown to improve accuracy of miniscrew placement, are too bulky and time consuming to become widely used in clinical practice, require customization, and often necessitate cone beam computed tomography, which is still unavailable in most settings. Many of these guides require individual fabrication in a laboratory setting and are associated with high fabrication expenses. The adhesive surgical guide as described herein requires little time, no additional radiographic exposure, no lab cost, and allows for same-day implant placement. The adhesive foil ring surgical guide can be utilized on any patient without any need for customization. As a result, the proposed guide design is faster and less expensive than many modern alternatives.

The guide will significantly reduce the learning curve and ameliorate clinician anxiety associated with placement of orthodontic miniscrews. Like any surgical procedure, outcomes of miniscrew placement are heavily dependent upon provider experience. Many orthodontists are uncomfortable with miniscrew placement and avoid their use entirely. Others choose to refer to oral surgeons or periodontists for surgical placement. This guide, if used properly, has the potential to reduce the risks involved with the procedure and could open the door for many orthodontists to begin utilizing TAD mechanics.

Some Exemplifications

Gingival adhesion removes the need for acrylic stents or tissue penetration in radiographic positioning. The exemplary guide has a nontoxic adhesive backing and can be adhered to the attached gingiva at the selected insertion site. The adhesive remains firmly attached to soft tissues of the mouth and is strong enough to resist movement due to incidental contact. The adhesive can be easily removed without damaging the underlying soft tissue with the application of water. Initial prototypes utilize a dry, cellulose-based adhesive.

Partially radiopaque guide ring allows visualization of the proposed implant site without completely obstructing underlying structures. The guide ring is radiopaque enough to be easily located on a radiograph while still allowing visualization of underlying structures including the lamina dura. This provides the clinician with more information regarding root proximity than could be provided by a completely radiopaque guide. Initial prototypes have achieved this optimal radiodensity using a 0.002″ thick stainless-steel foil. Stainless-steel is nontoxic and is already used in many orthodontic applications. The guide ring is designed to be as small as possible to avoid false positive and false negative readings, even with less than ideal orthoradial tube position. The ring has an inner diameter of 1.5 mm, allowing for 1.4 mm miniscrews to fit snugly without interference. The outer diameter is 3.5 mm, providing 2 mm of circumferential surface for adherence with the guide tube element while remaining small enough to minimize radiographic obstruction.

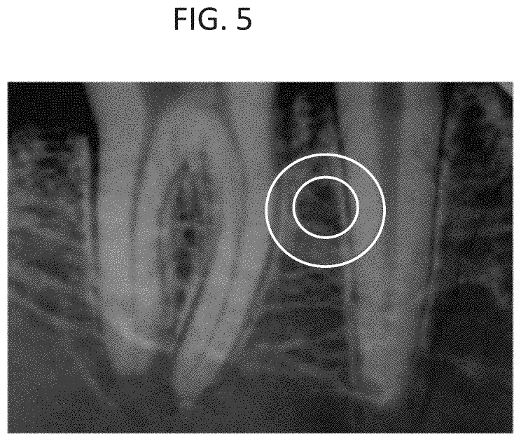

FIG. 5 is a radio image of a guide place on the gingiva of a subject. In FIG. 5 the outline of the guide is drawn over the image to more clearly indicate it's positioning.

Angulated guide tube allows for screw placement at the ideal angle to minimize both stresses and risk of root contact while maximizing insertable screw length. Most existing surgical guides are used to identify an insertion point but must be removed prior to screw placement. Because of this, these guides cannot help clinicians identify proper insertion angles. The exemplary guide has a guide tube that can remain in place for the initial insertion of the screw. Guide tubes are optimized according to insertion site and come in three variants. Maxillary guides have guide tubes positioned for miniscrew placement at 35° to the long axis of the teeth. Tubes on mandibular miniscrew guides are positioned for insertion at 15° to the long axis. Guides for placement near the maxillary sinus have tubes positioned perpendicular to the bony surface. These values are in accordance with current evidence-based recommendations. Guide tubes will be of a similar dimension to guide rings with an inner diameter of 1.5 mm and an outer diameter of 3.5 mm. Guide tubes will be firmly secured to guide rings with permanent adhesive. Guide tubes and be constructed with nontoxic acrylic or silicone tubing. Guide tubes can have an indicator mark to be aligned with the long axis of the teeth for angular calibration.

Vertical perforation allows removal after insertion through cortical plate. Guide tubes remain in place during initial insertion of the miniscrew to help guide angulation of insertion. Attached gingiva overlaying insertion sites averages 1 mm thick. Below this, the thickness of cortical bone is, in most cases, approximately 2 mm. This cortical bone provides a majority of the primary stability for miniscrews and angulation is maintainable following guide removal at that point. A guide tube height of 2.5 mm provides angular guidance while allowing a 6 mm miniscrew to be inserted fully through the cortical plate prior to removal of the guide. Both the guide tube and ring have a vertical perforation allowing the guide to be easily separated and removed once initial insertion has been completed. Following removal of the guide, the miniscrew may be inserted the remaining distance, maintaining the original angulation.

The miniscrews used in this study can be 1.4 mm diameter thread-forming VectorTAS™ miniscrews provided by Ormco™. 6 mm long miniscrews can be placed in anterior sites and 8 mm long miniscrews can be placed in posterior sites—according to manufacturer's instructions. A total of 100 miniscrews (experimental group, n=50; control group, n=50) can be placed. To assess interoperator variability, experimental and control groups can be evenly split between five clinicians. Miniscrews can be implanted into the maxillae and mandibles of 5 cadavers. Periapical radiographs can be exposed of each insertion site using a NOMAD Pro2™ portable X-ray device (Aribex™). In the control group, radiographs can be exposed with no guide. Insertion sites can be selected using periapical radiographs paired with clinical assessment to assess the safest point. In the experimental group, foil guides can be adhered to the attached gingiva at an insertion site chosen by clinical assessment alone. Guides can be adhered with the guide tubes at ideal angulation for miniscrew placement. Periapical radiographs can then be exposed from orthoradial tube position with the guides in place. Imaging of the guide can be used to assess the safety of the selected insertion site before proceeding. Miniscrews in both groups can then be inserted. In the control group, ideal angulation can be estimated clinically. In the experimental group, angulation can be determined by the surgical guide for initial insertion. Once the shoulder of the screw has reached the level of the guide tube, the guide can be split at the perforation and removed. The miniscrews can then be inserted the remainder of the way, maintaining their original angulations. A split-mouth approach can be used in selecting implantation sites, with quadrants assigned to either the experimental or control group, maintaining approximately equal number of right and left, maxillary and mandibular quadrants in each group and between each clinician. Five miniscrews can be placed in each quadrant, one in each interproximal space from lateral incisor to second molar. Following implantation, jaws can be sectioned and examined using microtomography. Root proximity can be assessed at three sites for each screw—at the neck of the screw, the midpoint, and the apex. Root damage and interradicular distance at each site can also be assessed using microtomography.

Statistical analysis. Each variable can be measured twice, by a single examiner, with two weeks separating measurements. Mean measurements can be recorded. Root proximities and interradicular distances for all groups can be reported as mean values with standard deviations. Interoperator variability can be assessed and compared for both groups. To assess differences in root proximity between groups, Wilcoxon 2-sample tests can be performed. Nonparametric rank sums will be used. Fisher exact tests can be used to assess significance at P<0.05

As used herein the term “comprising” or “comprises” is used in reference to compositions, methods, and respective component(s) thereof, that are essential to the claimed invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the claimed invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A surgical screw guide comprising,
a radio-translucent member extending in a first direction along a first axis from a first end to a second end,
the radio-translucent member including a channel extending along the first axis from the first end to the second end, and configured for guiding a screw therethrough,
the radio-translucent member including a radio-opaque portion adhered to the first end, wherein the radio-opaque portion includes an opening that is aligned with the channel.

2. The surgical guide according to claim 1, further comprising a tab consisting of a radio-translucent material attached between the first end of the elongated member and the radio-opaque portion.

3. The surgical guide as in claim 2, wherein the tab is configured as a flat member having a first surface attached to the first end of the radio-translucent member, a second surface attached to the radio-opaque portion, an opening that is aligned with the channel, and wherein the tab is configured not to adhere to biological tissue.

4. The surgical screw guide as in claim 1, wherein the surgical screw guide comprises at least three flat surfaces defining the channel and configured to engage screw flights of said screw for said guiding of the screw therethrough.

5. The surgical screw guide as in claim 4, wherein the channel is a closed channel defined by the at least three flat surfaces, the closed channel having an opening at the first end and an opening at the second end.

6. The surgical screw guide as in claim 1, further comprising an adhesive covering at least a portion of a flat exterior surface of the radio-opaque portion, wherein the adhesive and flat surface are configured for attachment of the radio-opaque portion to biological tissue.

7. The surgical screw guide as in claim 6, wherein the adhesive is an orthodontic grade adhesive.

8. The surgical screw guide as in claim 1, further comprising a slit extending through the length of the surgical screw guide in the first direction and wherein the slit extends through an outer surface of the radio-translucent member to the channel.

9. The surgical screw guide as in claim 1, wherein the radio-opaque portion includes a flat outer surface that is oriented at an angle relative to the first direction selected from the group consisting of about 90 degrees, about 105 degrees and about 125 degrees.

10. The surgical screw guide as in claim 1, wherein the radio-opaque portion attenuates more radiation than the rest of the radio-translucent member not including the radio-opaque portion, wherein the radiation is provided from a radiation beam oriented to irradiate in a direction approximately perpendicular to a flat outer surface of the radio-opaque portion and pass through the radio-translucent member.

11. The surgical screw guide as in claim 1, wherein the radio-opaque portion has a Hounsfield Unit (HU) greater than the HU of the radio-translucent member that is not the radio-opaque portion.

12. The surgical screw guide as in claim 1, wherein the radio-translucent member comprises a silicone polymer or an organic polymer.

13. The surgical screw guide as in claim 1, wherein the radio-opaque portion comprises a metal.

14. The surgical screw guide as in claim 14, wherein the metal is a stainless steel.

15. The surgical screw guide as in claim 1, wherein the radio-opaque portion is a stainless steel foil having a thickness between about 0.001 and 0.01 inches configured wherein the opening has a perimeter in the shape of a cylinder cross section.

16. The surgical screw guide as in claim 1, further comprising a miniscrew threaded into the channel, wherein the miniscrew has a diameter between 1 and 2 mm, and a length between 4 and 12 mm.

17. A surgical guide comprising;
a radio-opaque foil comprising a first surface and a second surface and an opening in the foil connecting the first surface and second surface, wherein at least a portion of the first surface comprises a film of orthodontic grade adhesive, and wherein the opening is configured for threading a miniscrew therethrough.

18. The surgical guide according to claim 17, further comprising a radio-translucent tube extending along a primary axis and having a first end and a second end and a channel extending between the first end and second end along the primary axis,
wherein the first end of the tube comprises a third surface and said third surface is attached to the second surface of the radio-opaque foil, wherein the opening in the foil is aligned to the channel.

19. The surgical guide according to claim 17, further comprising a tab constructed of a radio-translucent material attached to the second surface and extending outwards from the foil in a direction parallel to the first surface.

20. A method for providing orthodontic anchorage, the method comprising:
attaching a surgical guide to a subject;
the surgical guide comprising a radio-opaque foil comprising a first surface and a second surface and an opening in the foil connecting the first surface and second surface,
wherein attaching the surgical screw guide to the subject comprises contacting a gingiva of the subject with an adhesive, and contacting the adhesive with the first surface, and allowing the adhesive to cure;
irradiating the radio-opaque foil with an x-ray beam from an x-ray system, wherein the x-ray beams is oriented approximately perpendicular to the first and second surface of the foil;
detecting and processing x-rays transmitted through the radio-opaque foil using the x-ray system to provide an image of the radio-opaque foil relative to the teeth and jaw bone of said subject; and
feeding a screw through the screw guide of the surgical guide and fastening the screw into the jaw bone.
